(19) United States
(12) Patent Application Publication
(10) Pub. No.: US 2020/0138691 A1
(43) Pub. Date: May 7, 2020

(54) WATER-IN-OIL TYPE COSMETIC

(71) Applicant: FUJIFILM CORPORATION, Tokyo (JP)

(72) Inventors: Katsuhiko KANAZAWA, Tokyo (JP); Mayuko SUZUKI, Tokyo (JP); Ikuko OHGARU, Tokyo (JP); Toshiaki KUBO, Tokyo (JP)

(21) Appl. No.: 16/689,078

(22) Filed: Nov. 20, 2019

Related U.S. Application Data
(63) Continuation of application No. PCT/JP2018/019692, filed on May 22, 2018.

(30) Foreign Application Priority Data
May 25, 2017 (JP) 2017-103915

(51) Int. Cl. A61K 8/891; A61K 8/29; A61K 8/19; A61K 8/96; A61K 8/06; A61Q 17/04; A61Q 1/02

(52) U.S. Cl. CPC A61K 8/891; A61K 8/29; A61K 8/19; A61Q 1/02; A61K 8/064; A61Q 17/04; A61K 8/965

(57) ABSTRACT

Provided is a water-in-oil type cosmetic including: an oil agent having a melting point of 20° C. or lower; a silicone gel; a modified silicone surfactant having a hydrophile-lipophile balance value of less than 6; inorganic particles of at least one selected from the group consisting of titanium oxide, iron oxide, and mica; water; and a co-emulsifier, in which the oil agent having a melting point of 20° C. or lower contains 60% by mass or greater of silicone oil, a content of the modified silicone surfactant having a hydrophile-lipophile balance value of less than 6 is greater than 0.1% by mass and less than 1% by mass with respect to a total amount of the water-in-oil type cosmetic, and the inorganic particles of at least one selected from the group consisting of titanium oxide, iron oxide, and mica are contained in an oil phase.

### CROSS-REFERENCE TO RELATED APPLICATIONS

This application is a continuation application of International Application No. PCT/JP2018/019692, filed May 22, 2018, the disclosure of which is incorporated herein by reference in its entirety. Further, this application claims priority from Japanese Patent Application No. 2017-103915, filed May 25, 2017, the disclosure of which is incorporated herein by reference in its entirety.

### BACKGROUND OF THE INVENTION

### 1. Field of the Invention

The present disclosure relates to a water-in-oil type cosmetic.

### 2. Description of the Related Art

Water-in-oil type cosmetics having an oil component as a continuous phase have been widely known. The water-in-oil type cosmetics moisturize the skin, but there is a problem in that it is easy to feel stickiness and difficult to feel freshness.

Therefore, an attempt to obtain freshness which is a water overflowing feeling even in a case of using a water-in-oil type cosmetic has been made using a phenomenon in which an emulsified state of a water-in-oil type cosmetic is broken due to the shear force at the time of application of the cosmetic onto the skin and water in the dispersed phase is released onto the skin.

As a water-in-oil type cosmetic which can provide a feeling of use that water splashes at the time of being applied onto the skin, for example, JP2011-219448A discloses a water-releasing makeup cosmetic formed of a water-in-oil type cosmetic containing (a) partially crosslinked polyether-modified silicone, partially crosslinked polyglycerin-modified silicone, or a combination of these, (b) acryl-silicone-based graft copolymer, and 0.05% to 0.7% by weight of (c) branched silicone surfactant.

Further, as a water-in-oil type cosmetic which can provide an excellent water overflowing feeling, for example, WO2013/136616A discloses a water-in-oil type emulsion composition in which a combination of 3,7,11,15-tetramethyl-1,2,3-hexadecanetriol, diglyceryl diisostearate, and a polyoxyethylene-methyl polysiloxane copolymer is used as an emulsifier.

### SUMMARY OF THE INVENTION

It has been widely known that inorganic particles are used in cosmetics for the purpose of adjusting the skin color, exhibiting the covering powder, and diffusing ultraviolet rays.

As a result of examination conducted on the water-in-oil type cosmetic containing inorganic particles which has been prepared using the technique disclosed in JP2011-219448A or WO2013/136616A, the present inventors found that so-called coating unevenness occurs since the inorganic particles do not uniformly spread on the skin at the time of application of the cosmetic onto the skin even though the water overflowing feeling at the time of application of the cosmetic onto the skin is maintained, and emulsion stability of the water-in-oil type cosmetic over time is degraded.

The present invention has been made in consideration of the above-described circumstances, and an object of the present invention is to provide a water-in-oil type cosmetic which contain inorganic particles and has an excellent emulsifying property and excellent emulsion stability and in which coating unevenness is reduced while the water overflowing feeling at the time of application of the cosmetic onto the skin is excellent.

Specific means for solving the above-described problems include the following embodiments.

<1> A water-in-oil type cosmetic comprising: an oil agent having a melting point of 20° C. or lower; a silicone gel; a modified silicone surfactant having a hydrophile-lipophile balance value of less than 6; inorganic particles of at least one selected from the group consisting of titanium oxide, iron oxide, and mica; water; and a co-emulsifier, wherein the oil agent having a melting point of 20° C. or lower comprises 60% by mass or greater of silicone oil, a content of the modified silicone surfactant having a hydrophile-lipophile balance value of less than 6 is greater than 0.1% by mass and less than 1% by mass with respect to a total amount of the water-in-oil type cosmetic, and the inorganic particles of at least one selected from the group consisting of titanium oxide, iron oxide, and mica are contained in an oil phase.

<2> The water-in-oil type cosmetic according to <1>, wherein the oil phase comprises the inorganic particles of at least one selected from the group consisting of titanium oxide, iron oxide, and mica at a content of 5% by mass to 30% by mass with respect to the total amount of the water-in-oil type cosmetic.

<3> The water-in-oil type cosmetic according to <1> or <2>, having a water phase comprising solid particles.

<4> The water-in-oil type cosmetic according to <3>, wherein the solid particles are hydrophobic solid particles a surface of each of which has been subjected to a hydrophilic treatment or hydrophobic solid particles to which dispersibility in the water phase has been imparted.

<5> The water-in-oil type cosmetic according to any one of <1> to <4>, wherein the modified silicone surfactant having a hydrophile-lipophile balance value of less than 6 has a branched silicone chain in a structure thereof.

<6> The water-in-oil type cosmetic according to any one of <1> to <5>, wherein the water phase comprises a thickening polysaccharide.

<7> The water-in-oil type cosmetic according to any one of <1> to <6>, wherein the co-emulsifier is at least one compound selected from the group consisting of a compound represented by Formula (1) and a compound represented by Formula (2):

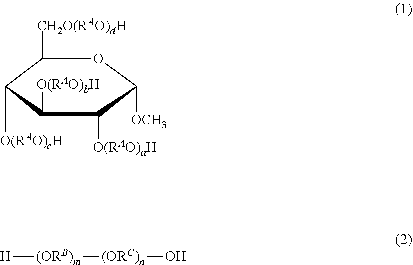

In Formula (1), R^{A} represents —CH₂CH₂— or —CH₂CH₂CH₂—; a, b, c, and d respectively represent an average addition molar number of (R^{A}O) and are each independently in a range of 0 to 200; and a+b+c+d is in a range of 3 to 200.

In Formula (2), R^{B} represents —CH₂CH₂—; R^{C} represents —CH₂CH₂CH₂—; m represents an average addition molar number of (OR^{B}); n represents an average addition molar number of (OR^{C}); n and m are each independently in a range of 0 to 200; and m+n is in a range of 3 to 200.

<8>

The water-in-oil type cosmetic according to any one of <1> to <6>, wherein the co-emulsifier is at least one compound selected from the group consisting of a compound represented by Formula (2).

H—(OR^{B})ₘ—(OR^{C})ₙ—OH (2)

In Formula (2), R^{B} represents —CH₂CH₂—; R^{C} represents or —CH₂CH₂CH₂—; m represents an average addition molar number of (OR^{B}); n represents an average addition molar number of (OR^{C}); n and m are each independently in a range of 0 to 200; and m+n is in a range of 3 to 200.

<9> The water-in-oil type cosmetic according to <8>, wherein, in the compound represented by Formula (2), m+n is in a range of 6 to 75.

<10> The water-in-oil type cosmetic according to any one of <1> to <9>, wherein the silicone oil comprises at least one selected from the group consisting of dimethylpolysiloxane and decamethylcyclopentasiloxane.

<11> The water-in-oil type cosmetic according to any one of <1> to <10>, wherein the oil agent having a melting point of 20° C. or lower comprises 75% by mass to 100% by mass of the silicone oil.

<12> The water-in-oil type cosmetic according to any one of <1> to <11>, wherein the silicone gel comprises at least one selected from the group consisting of a polyether-modified silicone gel and a silicone three-dimensional crosslinked product.

<13> The water-in-oil type cosmetic according to any one of <1> to <12>, wherein a content of the silicone gel is in a range of 0.25% by mass to 4.5% by mass with respect to the total amount of the water-in-oil type cosmetic.

<14> The water-in-oil type cosmetic according to any one of <1> to <13>, having a viscosity at 25° C. of 3000 mPa·s or greater.

According to an embodiment of the present invention, it is possible to provide a water-in-oil type cosmetic which contain inorganic particles and has an excellent emulsifying property and excellent emulsion stability and in which coating unevenness is reduced while the water overflowing feeling at the time of application of the cosmetic onto the skin is excellent.

### DESCRIPTION OF THE PREFERRED EMBODIMENTS

Hereinafter, embodiments of a water-in-oil type cosmetic to which the present invention has been applied will be described. Here, the present invention is not limited to the following embodiments, and modifications can be made as appropriate within the range not departing from the purpose of the present invention.

In the present disclosure, the numerical ranges shown using “to” indicate ranges including the numerical values described before and after “to” as the minimum values and the maximum values.

In the numerical ranges described in a stepwise manner in the present disclosure, the upper limits or the lower limits described in certain numerical ranges may be replaced with the upper limits or the lower limits in other numerical ranges described in a stepwise manner. Further, in the numerical ranges described in the present specification, the upper limits or the lower limits described in certain numerical ranges may be replaced with values described in examples.

In the present disclosure, the amount of each component in a water-in-oil type cosmetic indicates the total amount of a plurality of kinds of materials present in the water-in-oil type cosmetic unless otherwise specified in a case where a plurality of kinds of materials corresponding each component are present in the water-in-oil type cosmetic.

In the present disclosure, the “oil phase” indicates a continuous phase of the water-in-oil type cosmetic and contains a liquid medium of the continuous phase and a component dispersed or dissolved in the liquid medium.

In the present disclosure, the “water phase” indicates a dispersed phase in the water-in-oil type cosmetic and contains a liquid medium of the dispersed phase and a component dispersed or dissolved in the liquid medium.

<Water-in-Oil Type Cosmetic>

A water-in-oil type cosmetic according to the present embodiment includes (A) an oil agent having a melting point of 20° C. or lower, (B) silicone gel, (C) a modified silicone surfactant having an HLB value of less than 6, (D) inorganic particles of at least one selected from the group consisting of titanium oxide, iron oxide, and mica, (E) water, and (F) a co-emulsifier.

Further, in the water-in-oil type cosmetic according to the present embodiment, the (A) oil agent having a melting point of 20° C. or lower contains 60% by mass or greater of silicone oil, the content of the (C) modified silicone surfactant having a hydrophile-lipophile balance value of less than 6 is greater than 0.1% by mass and less than 1% by mass with respect to the total amount of the water-in-oil type cosmetic, and (D) inorganic particles of at least one selected from the group consisting of titanium oxide, iron oxide, and mica is contained in an oil phase.

In the present disclosure, the oil agent having a melting point of 20° C. or lower is also referred to as the “(A) oil agent, the (C) modified silicone surfactant having a hydrophile-lipophile balance value of less than 6 is also referred to as the “(C) modified silicone surfactant”, and (D) inorganic particles of at least one selected from the group consisting of titanium oxide, iron oxide, and mica is also referred to as the “(D) inorganic particles” as appropriate.

Further, the hydrophile-lipophile balance value of the (C) modified silicone surfactant is also abbreviated as the HLB (hydrophile-lipophile balance) value.

With the above-described configuration, the water-in-oil type cosmetic according to the present embodiment has an excellent emulsifying property and excellent emulsion stability while containing inorganic particles and has reduced coating unevenness while the water overflowing feeling at the time of application of the cosmetic onto the skin is excellent. As described above, the reason why the emulsifying property and the emulsion stability of the water-in-oil type cosmetic according to the present embodiment are excellent, the water overflowing feeling at the time of application of the cosmetic onto the skin is excellent, and the coating unevenness is reduced is assumed as follows, but the present invention is not limited thereto.

The water-in-oil type cosmetic according to the present embodiment includes the (A) oil agent having a melting point of 20° C. or lower, the (B) silicone gel, and greater than 0.1% by mass and less than 1% by mass of the (C) modified silicone surfactant having an HLB value of less than 6 with respect to the total amount of the water-in-oil type cosmetic, and the (A) oil agent having a melting point of 20° C. or lower contains 60% by mass or greater of the silicone oil. Since the components (B) and (C) have a silicone chain similarly to silicone oil, the (B) silicone gel tends to be swollen in silicone oil, and the (C) modified silicone surfactant tends to be dissolved or dispersed in silicone oil. The water-in-oil type cosmetic containing the (D) inorganic particles in an oil phase is prepared by employing the configuration (the balance between the presence of each component and the content thereof) of the water-in-oil type cosmetic according to the present embodiment while having such mutual relationships. As the result, a water-in-oil type cosmetic having an emulsion with improved temporal stability and having an excellent water overflowing feeling at the time of being applied onto the skin is obtained. It is considered that the water overflowing feeling at the time of application of the cosmetic onto the skin becomes excellent since the emulsified state of the water-in-oil type cosmetic according to the present embodiment is easily broken due to the shear force applied at the time of application of the cosmetic onto the skin.

Further, the water-in-oil type cosmetic according to the present embodiment contains the (F) co-emulsifier in a water phase of the water-in-oil type cosmetic. The (F) co-emulsifier does not almost act on the temporal stability of the emulsion and improves the familiarity between the oil phase and the water phase in application of the cosmetic onto the skin or the drying process after the application of the cosmetic onto the skin. Therefore, it is considered that the coating unevenness is suppressed in a case of using the water-in-oil type cosmetic according to the present embodiment.

Here, in the present disclosure, the “emulsifying property is excellent” indicates that the water-in-oil type cosmetic containing the (D) inorganic particles is prepared and the prepared water-in-oil type cosmetic is not separated within 3 days or the water-in-oil type cosmetic containing the (D) inorganic particles is prepared and an oil phase component does not float (liquid separation).

Further, the “separation” indicates that the prepared water-in-oil type cosmetic is divided into an oil phase and a water phase. Further, the “liquid separation” indicates that distribution of the oil phase component becomes uneven (for example, the oil agent and powder are separated due to the specific gravity or the like) in the oil phase of the prepared water-in-oil type cosmetic.

The “emulsion stability” indicates that the temporal stability of the prepared water-in-oil type cosmetic is excellent, in other words, the emulsified state of the prepared water-in-oil type cosmetic is maintained for 1 week or longer. Further, evaluation may be performed using an acceleration test or a severe test in order to predict the state of the water-in-oil type cosmetic over time.

The “water overflowing feeling is excellent” indicates that the feeling that water droplets appear can be recognized at the time of application (applying and spreading) of the prepared water-in-oil type cosmetic onto the skin.

The “coating unevenness” indicates the state in which the locality (non-uniform distribution) described below can be visually recognized. The water phase component spreads on the surface of the skin due to unification or breakage of the emulsified state of the water phase particles at the time of application (applying and spreading) of the prepared water-in-oil type cosmetic onto the skin. In this manner, since the water phase component is firstly applied and spreads on the skin, the oil phase which is the outer phase of the water-in-oil type cosmetic repels on the skin. As the result, locality of the oil phase or the water phase occurs. Particularly, in a case where the oil phase component contains a pigment or the like as a color developing component, the coating unevenness becomes significant on the skin and also affects the merchantability, which is not preferable.

Hereinafter, the components which can be contained in the water-in-oil type cosmetic according to the present embodiment will be described in detail.

[(A) Oil Agent Having Melting Point of 20° C. or Lower]

The water-in-oil type cosmetic according to the present embodiment contains the (A) oil agent having a melting point of 20° C. or lower.

The (A) oil agent having a melting point of 20° C. or lower is a component which becomes a solvent or a dispersion medium in the composition forming the oil phase of the water-in-oil type cosmetic.

In the present embodiment, the (A) oil agent having a melting point of 20° C. or lower indicates an oil agent that enters a liquid state at room temperature (25° C.).

The viscosity of the (A) oil agent at room temperature (25° C.) is preferably in a range of 1 mPa·s to 300 mPa·s. Further, from the viewpoints of spreading the water-in-oil type cosmetic on the skin at the time of application of the cosmetic and the stickiness at the time of application of the cosmetic is more preferably in a range of 1 mPa·s to 50 mPa·s and still more preferably in a range of 1 mPa·s to 30 mPa·s.

Here, in the present disclosure, the viscosity of the (A) oil agent at room temperature (25° C.) can be measured according to a known measuring method, but a value measured using the same measurement principle as that for the viscosity of the water-in-oil type cosmetic described below at 25° C. can be used. Further, the rotor, the rotation speed, the rotation time, and the like can be appropriately and properly changed according to the viscosity.

In the present embodiment, the (A) oil agent contains 60% by mass or greater of the silicone oil.

The (A) oil agent may contain only one or two or more kinds of silicone oils.

The content of the silicone oil (in other words, the ratio of the mass of the silicone oil to the total mass of the (A) oil agent) may be 60% by mass or greater with respect to the mass of the (A) oil agent. Further, as the content of the silicone oil increases, since the compatibility of the silicone oil with the (C) modified silicone surfactant or the (B) silicone gel or the dispersibility of the (C) modified silicone surfactant or the (B) silicone gel becomes excellent, the content thereof is preferably 65% by mass or greater, more preferably 75% by mass or greater, still more preferably 80% by mass or greater, and even still more preferably 90% by mass or greater. The entire (A) oil agent may be silicone oil (in other words, the content of the silicone oil in the (A) oil agent is 100% by mass).

According to the present embodiment, the content of the silicone oil in the (A) oil agent is preferably in a range of 75% by mass to 100% by mass.

Examples of the silicone oil include chain polysiloxane such as dimethyl polysiloxane (dimethicone), methyl phenyl polysiloxane, methyl hydrogen polysiloxane, diphenyl siloxy phenyl trimethicone, or methyl trimethicone; cyclic polysiloxane such as octamethyl cyclotetrasiloxane, decamethyl cyclopentasiloxane (cyclopentasiloxane), dodecamethyl cyclohexane siloxane, tetramethyl tetrahydrogen cyclotetrasiloxane; and caprylyl methicone.

Examples of commercially available products of the silicone oil include KF-96L-0.65cs, KF-96L-1cs, KF-96L-1.5cs, KF-96L-2cs, KF-96L-5cs, KF-96A-6cs, KF-96-10cs, KF-96-20cs, and KF-995 (all manufactured by Shin-Etsu Chemical Co., Ltd.), SH200 C Fluid 1CS, SH200 Fluid 1.5CS, SH200 C 2CS, SH200 C Fluid 5CS, SH200 C Fluid 6CS, SH200 C Fluid 10CS, SH200 C Fluid 20CS, 2-1184 Fluid, SH245 Fluid, DC246 Fluid, DC345 Fluid, and SS-3408 (all manufactured by Dow Corning Toray Co., Ltd.), and TSF404, TSF405, and TSF4045 (all manufactured by Momentive Performance Materials Inc.).

Among the examples of the silicone oil, from the viewpoints of the availability, the swelling property of the (B) silicone gel, the solubility of the (C) modified silicone surfactant having an HLB value of less than 6, and the emulsion stability, at least one selected from the group consisting of dimethyl polysiloxane (dimethicone) and decamethyl cyclopentasiloxane (cyclopentasiloxane).

Particularly, from the viewpoint of improving the emulsion stability, a combination of dimethyl polysiloxane (dimethicone) and decamethyl cyclopentasiloxane (cyclopentasiloxane) is preferable as the silicone oil.

Examples of the oil agent which may constitute the (A) oil agent together with the silicone oil include ester oil and hydrocarbon oil.

The (A) oil agent may include only one or two or more kinds of oil agents other than the silicone oil.

In a case where the water-in-oil type cosmetic according to the present embodiment contains an organic ultraviolet absorbing agent, from the viewpoint of the solubility of the organic ultraviolet absorbing agent, it is preferable to use ester oil.

Examples of the ester oil include pentaerythrityl tetraethyl hexanoate, cetyl ethyl hexanoate, jojoba oil, di(phytosteryl/octyldodecyl) lauroyl glutamate, triisostearin, glyceryl diisostearate, triethyl hexanoin, (phytosteryl/behenyl) dimer dilinoleate, (phytosteryl/isostearyl/cetyl/stearyl/behenyl) dimer dilinoleate, isopropyl palmitate, macadamia nut fatty acid phytosteryl, tetra(behenic acid/benzoic acid/ethylhexanoic acid) pentaerythrityl, ethylhexyl palmitate, myristyl myristate, tripropylene glycol dipivalate, and isotridecyl isononanoate.

The hydrocarbon oil may be any of linear hydrocarbon oil or branched hydrocarbon oil.

Examples of the hydrocarbon oil include isoalkane (isoparaffin) having 8 to 16 carbon atoms such as isodecane, isododecane, or isohexadecane, mineral oil, and squalane.

The content of the (A) oil agent in the water-in-oil type cosmetic (in other words, the ratio of the mass of the (A) oil agent to the total mass of the water-in-oil type cosmetic) is preferably in a range of 1% by mass to 40% by mass, more preferably in a range of 5% by mass to 35% by mass, still more preferably in a range of 10% by mass to 30% by mass, and even still more preferably in a range of 12% by mass to 30% by mass with respect to the total amount of the water-in-oil type cosmetic.

In addition, the content of the (A) oil agent includes the content of oil contained together with the silicone gel in a case of using a commercially available product of the (B) silicone gel described below at the time of production of the water-in-oil type cosmetic according to the present embodiment.

[(B) Silicone Gel]

The water-in-oil type cosmetic according to the present embodiment contains the (B) silicone gel.

The (B) silicone gel in the present disclosure indicates a silicone crosslinked product in which silicone chains forming the main chain skeleton are crosslinked by a polyether chain, a polyglycerin chain, and a silicone chain.

Based on the structure of the chain used for crosslinking, examples of the kind of the (B) silicone gel include polyether-modified silicone gel, polyglycerin-modified silicone gel, and a silicone three-dimensional crosslinked product (in other words, a silicone crosslinked product in which silicone chains forming the main chain skeleton are crosslinked by a silicone chain). These can be used for the (B) silicone gel according to the present embodiment without limitation.

The polyether-modified silicone gel, the polyglycerin-modified silicone gel, and the silicone three-dimensional crosslinked product may have an alkyl chain as a branched chain in the silicone chain forming the main chain skeleton or may have an alkyl chain and a silicone chain.

From the viewpoint of the swelling property of the (B) silicone gel with respect to the (A) oil agent containing silicone oil, it is preferable that the (B) silicone gel contains at least one selected from the group consisting of polyether-modified silicone gel and a silicone three-dimensional crosslinked product and more preferable that the (B) silicone gel contains polyether-modified silicone gel and a silicone three-dimensional crosslinked product.

Specific examples of the silicone gel include a (dimethicone/(PEG-10/15)) cross polymer, a (PEG-15/lauryldimethicone) cross polymer, a (PEG-10/lauryldimethicone) cross polymer, a (PEG-15/lauryl polydimethyl siloxyethyldimethicone) cross polymer, a (dimethicone/polyglycerin-3) cross polymer, a (lauryldimethicone/polyglycerin-3) cross polymer, a (polyglyceryl-3/laurylpolydimethylsiloxyethyldimethicone) cross polymer, a (dimethicone/vinyldimethicone) cross polymer, a (dimethicone/phenylvinyldimethicone) cross polymer, a (vinyldimethicone/lauryldimethicone) cross polymer, and a (laurylpolydimethylsiloxyethyldimethicone/bisvinyldimethicone) cross polymer.

Examples of commercially available products of the silicone gel include KSG-210, KSG-240, KSG-310, KSG-320, KSG-330, KSG-340, KSG-320Z, KSG-350Z, KSG-360Z, KSG-380Z, KSG-710, KSG-810, KSG-820, KSG-830, KSG-840, KSG-820Z, KSG-850Z, KSG-15, KSG-1510, KSG-16, KSG-1610, KSG-18A, KSG-19, KSG-016F, KSG-41A, KSG-42A, KSG-43A, KSG-44A, KSG-042Z, KSG-045Z, and KSG-048Z (all manufactured by Shin-Etsu Chemical Co., Ltd.).

From the viewpoints of the availability, the swelling property of the (B) silicone gel with respect to the (A) oil agent containing silicone oil, and the emulsion stability, it is preferable that the (B) silicone gel contains at least one selected from the group consisting of a (dimethicone/(PEG-10/15) cross polymer and a (dimethicone/vinyldimethicone) cross polymer and more preferable that the (B) silicone gel contains a (dimethicone/(PEG-10/15) cross polymer and a (dimethicone/vinyldimethicone) cross polymer.

The content of the (B) silicone gel in the water-in-oil type cosmetic (in other words, the ratio of the mass of the (B) silicone gel to the total mass of the water-in-oil type cosmetic) is preferably in a range of 0.1% by mass to 8% by mass, more preferably in a range of 0.25% by mass to 4.5% by mass, and still more preferably in a range of 0.5% by mass to 3% by mass with respect to the total amount of the water-in-oil type cosmetic.

The water-in-oil type cosmetic according to the present embodiment may contain only one or two or more kinds of silicone gels (B).

[(C) Modified Silicone Surfactant Having HLB Value of Less than 6]

The water-in-oil type cosmetic according to the present embodiment contains the (C) modified silicone surfactant having an HLB value of less than 6.

The (C) modified silicone surfactant indicates a silicone compound in which silicone chains forming the main chain skeleton are not crosslinked and which is modified by a hydrophilic organic group.

Depending on the structure of the hydrophilic organic group used for modification, examples of the (C) modified silicone surfactant include a polyether-modified silicone surfactant, a polyglycerin-modified silicone surfactant, a polyether-alkyl co-modified silicone surfactant, and a polyglycerin-alkyl co-modified silicone surfactant. These can be used without limitation as long as the (C) modified silicone surfactant according to the present embodiment has an HLB value of less than 6.

Further, as the (C) modified silicone surfactant, the silicone chain forming the main chain skeleton may be linear or branched.

Among the examples of the (C) modified silicone surfactant, from the viewpoint of the emulsifying property, a polyether-modified silicone surfactant and a polyether-alkyl co-modified silicone surfactant are preferable, and a polyether-modified silicone surfactant is more preferable.

Further, among the examples of the (C) modified silicone surfactant, from the viewpoint of the emulsion stability, a modified silicone surfactant whose silicone chain forming the main chain skeleton is branched, in other words, a surfactant which has a branched silicone chain in the structure is preferable (that is, modified silicone which has a branched silicone chain in the structure), and polyether-modified silicone which has a branched silicone chain in the structure is particularly preferable.

The HLB value of the (C) modified silicone surfactant is less than 6 from the viewpoint of producing the water-in-oil type cosmetic and is preferably 1 or greater from the viewpoints of the availability and easily producing the water-in-oil type cosmetic.

From the viewpoint of the emulsion stability of the water-in-oil type cosmetic, the HLB value of the (C) modified silicone surfactant is more preferably 1 or greater and less than 6, still more preferably 2 or greater and less than 6, and particularly preferably in a range of 2 to 5.

In the present disclosure, the HLB value of the (C) modified silicone surfactant is a value acquired from the molecular weight and the number of hydrophilic organic groups such as an ethylene oxide group. Further, in a case of using a commercially available product, the HLB value described in the catalog or the like can be employed.

Specific examples of the (C) modified silicone surfactant include PEG-3 dimethicone, PEG-9 methyl ether dimethicone, PEG-10 dimethicone, PEG-9 polydimethylsiloxyethyl dimethicone, lauryl PEG-9 polydimethylsiloxyethyl dimethicone, cetyl PEG/PPG-10/1 dimethicone, polyglyceryl-3 polydimethylsiloxyethyl dimethicone, and lauryl polyglyceryl-3 polydimethylsiloxyethyl dimethicone.

Among these, the modified silicone having a branched silicone chain in the structure corresponds to PEG-9 polydimethylsiloxyethyl dimethicone, lauryl PEG-9 polydimethylsiloxyethyl dimethicone, polyglyceryl-3 polydimethylsiloxyethyl dimethicone, and lauryl polyglyceryl-3 polydimethylsiloxyethyl dimethicone. Among these, the polyether-modified silicone having a branched silicone chain in the structure corresponds to PEG-9 polydimethylsiloxyethyl dimethicone.

Examples of commercially available products of the (C) modified silicone surfactant include KF-6015, KF-6016, KF-6017, KF-6017P, KF-6028, KF-6028P, KF-6038, and KF-6048 (all manufactured by Shin-Etsu Chemical Co., Ltd.).

Among the examples of the (C) modified silicone surfactant, from the viewpoint of improving the emulsion stability and the water overflowing feeling at the time of application of the cosmetic onto the skin, at least one selected from PEG-9 polydimethylsiloxyethyl dimethicone or lauryl PEG-9 polydimethylsiloxyethyl dimethicone is preferable.

From the viewpoints of improving the emulsifying property and the emulsion stability and obtaining the water overflowing feeling at the time of application onto the skin, the content of the (C) modified silicone surfactant (in other words, the ratio of the mass of the (C) modified silicone surfactant to the total mass of the water-in-oil type cosmetic) is greater than 0.1% by mass and less than 1% by mass, preferably in a range of 0.2% by mass to 0.8% by mass, and still more preferably in a range of 0.3% by mass to 0.7% by mass with respect to the total amount of the water-in-oil type cosmetic.

The water-in-oil type cosmetic according to the present embodiment may contain only one or two or more kinds of the (C) modified silicone surfactant.

[(D) Inorganic Particles of at Least One Selected from Group Consisting of Titanium Oxide, Iron Oxide, and Mica]

The water-in-oil type cosmetic according to the present embodiment contains (D) inorganic particles of at least one selected from the group consisting of titanium oxide, iron oxide, and mica in the oil phase thereof.

Here, the “(D) inorganic particles are contained in the oil phase” indicates that the (D) inorganic particles are present in the oil phase which is a continuous phase. It is preferable that the (D) inorganic particles are dispersed and contained in the oil phase in order to easily exhibit the function of the (D) inorganic particles.

The (D) inorganic particles may be appropriately selected according to the applications of the water-in-oil type cosmetic according to the present embodiment and the compatibility with the (A) oil agent, and the water-in-oil type cosmetic may contain only one or two or more kinds of (D) inorganic particles.

Further, the surface of each (D) inorganic particle may be subjected to a hydrophobic treatment.

Titanium oxide is an inorganic particle used for a white coloring material pigment, a shielding agent for exhibiting covering power, or an ultraviolet diffusion agent.

The titanium oxide according to the present embodiment is not particularly limited as long as the titanium oxide which can be applied to the cosmetic and may be fine particle titanium oxide or pigment grade titanium oxide.

Further, the fine particle titanium oxide used in the cosmetic indicates titanium oxide having a primary particle diameter of several nanometers to several tens of nanometers, and the pigment grade titanium oxide indicates titanium oxide having a particle diameter of several hundreds of nanometers.

In the present disclosure, the particle diameter of the (D) inorganic particles can be acquired by analyzing an image of an electron microscope such as a transmission electron microscope. In a case of using a commercially available product as the inorganic particle, the value described in the catalog or the like can be employed.

Here, from the viewpoints of easy introduction into the oil phase and the emulsion stability, titanium oxide whose surface is subjected to a hydrophobic treatment (also referred to as surface-hydrophobicized titanium oxide) is preferable.

Specific examples of the titanium oxide include those described in paragraphs 0019 to 0030 of JP2017-031380A or commercially available products.

Examples of the commercially available products of the titanium oxide include, as the titanium oxide whose surface is subjected to a hydrophobic treatment (surface-hydrophobicized titanium oxide), OTS-2 TiO₂ CR-50, SI06 TiO₂, TTO-55, and SI06 TiO₂ CR-50 (all manufactured by Daito Kasei Kogyo Co., Ltd.), and HXMT-100ZA (manufactured by Tayca Corporation).

Here, HXMT-100ZA is titanium oxide whose surface is treated with aluminum hydroxide and stearic acid and then further treated with 4-tert-butyl-4′-methoxydibenzoylmethane (t-butylmethoxydibenzoylmethane).

Iron oxide is one of coloring material pigments used for adjusting the skin color.

Examples of the iron oxide according to the present embodiment include yellow iron oxide, red iron oxide, and black iron oxide, and a mixture of these is preferable.

Specific examples of the iron oxide include commercially available products such as OTS-2 RED R-516L (red iron oxide), OTS-2 BLACK BL-100 (black iron oxide), and OTS-2 YELLOW LLXLO (yellow iron oxide) (all manufactured by Daito Kasei Kogyo Co., Ltd.) as iron oxide whose surface is subjected to a hydrophobic treatment.

Mica is an inorganic particle used for an extender pigment.

As the mica according to the present embodiment, both of natural mica and synthetic mica can be used.

Specific examples of the mica include commercially available products such as SERICITE FSE (manufactured by Sanshin Mining Ind. Co., Ltd.) and Synthetic Phlogopite PDM Series (manufactured by Topy Industries, Ltd.).

Further, the mica is used as a pearl pigment by being coated with titanium oxide, iron oxide, or the like.

In the present embodiment, the mica being coated with titanium oxide, iron oxide, or the like is also contained in the (D) inorganic particles.

The content of the (D) inorganic particles contained in the oil phase (in other words, the ratio of the mass of the (D) inorganic particles contained in the oil phase to the total mass of the water-in-oil type cosmetic) may be determined depending on the applications of the water-in-oil type cosmetic. The content of the (D) inorganic particles contained in the oil phase is preferably in a range of 3% by mass to 30% by mass with respect to the total amount of the water-in-oil type cosmetic from the viewpoints of exhibiting the function obtained by addition of the inorganic particles and the emulsion stability, more preferably in a range of 4.5% by mass to 20% by mass from the viewpoint of the emulsion stability, and still more preferably in a range of 6% by mass to 20% by mass from the viewpoints of exhibiting the covering power, adjusting the skin color, and diffusibility of ultraviolet rays.

Further, the above-described content indicates only the content of the inorganic particles contained in the oil phase. Even in a case where titanium oxide is contained in the water phase, the amount of the titanium oxide in the water phase is not included in the above-described content.

In the present disclosure, a method of verifying the presence of the (D) inorganic particles in the oil phase and the method of measuring the content of the (D) inorganic particles in the oil phase are as follows.

In other words, the presence of the (D) inorganic particles in the oil phase and the content thereof are acquired by centrifuging the measurement sample (that is, the water-in-oil type cosmetic), performing elemental analysis on the layer obtained by separation or extraction through the centrifugation, and performing analysis through comparison with the components (the component display of the cosmetic may be used) used for formulation of the measurement sample based on the results thereof. During the measurement, the measurement sample may be diluted with an organic solvent or water, and means required for the analysis such as a treatment or step of removing components other than the (D) inorganic particles may be appropriately used.

[(E) Water]

The water-in-oil type cosmetic according to the present embodiment contains (E) water.

The (E) water is a component which becomes a solvent or a dispersion medium in the composition forming the water phase of the water-in-oil type cosmetic.

The (E) water is not particularly limited as long as the water can be applied to the cosmetic.

[(F) Co-Emulsifier]

The water-in-oil type cosmetic according to the present embodiment contains the (F) co-emulsifier.

The (F) co-emulsifier according to the present embodiment is not a compound that directly participates in formation of the emulsified state, but a compound which is dissolved in the water phase, does not almost act on the emulsion stability of the water-in-oil type cosmetic, and contributes to familiarity between the oil phase and the water phase in the drying process at the time of application of the cosmetic onto the skin or after the application of the cosmetic onto the skin.

Further, the (F) co-emulsifier does not contain polyhydric alcohol such as glycerin or 1,3-butylene glycol because of the following reason.

In other words, glycerin is generally known to contribute emulsion stabilization in an oil-in-water type cosmetic, but the present embodiment relates to a water-in-oil type cosmetic. Therefore, glycerin is not contained in the co-emulsifier. Further, since diols such as 1,3-butylene glycol function as a solubilizing agent, emulsion destabilization is caused. Therefore, diols such as 1,3-butylene glycol is not contained in the co-emulsifier.

Examples of the (F) co-emulsifier include amino acids and derivatives thereof, peptides having a molecular weight of 500 or less, saccharides having a molecular weight of 5000 or less and derivatives thereof, polyethylene glycol, polypropylene glycol, and derivatives thereof, and amphoteric compounds having a molecular weight of 500 or less such as betaine.

From the viewpoints of exhibiting the solubility in water and the effect of improving the coating unevenness, and suitability to a living body, at least one compound selected from the group consisting of a compound represented by Formula (1) and a compound represented by formula (2) is preferable as the (F) co-emulsifier.

These compounds are compounds which contain at least one of “—CH₂CH₂—O— (PEO chain: polyethylene oxide chain)” or “—CH₂CH₂CH₂—O— (PPO chain: polypropylene oxide chain)” and the molecular terminal group has a plurality of structures containing one or more of “OH”.

First, the compound represented by Formula (1) will be described.

The compound represented by formula (1) is a compound obtained by adding a (polyethylene oxide group) PEO group or a (polypropylene oxide group) PPO group to methyl glycoside.

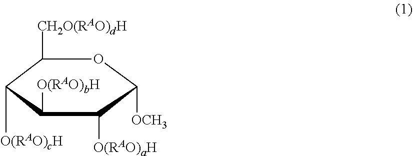

In Formula (1), R^{A} represents —CH₂CH₂— or —CH₂CH₂CH₂—, a, b, c, and d respectively representing an average addition molar number of (R^{A}O) are each independently in a range of 0 to 200, and a+b+c+d is in a range of 3 to 200.

In a case where a+b+c+d in Formula (1) is in a range of 5 to 50, the compound is in a liquid state at room temperature (25° C.) and has excellent handleability and thus heating or melting is not required, which is preferable.

Further, from the viewpoints of excellent availability of the compound and excellent applicability to the skin, a+b+c+d is preferably in a range of 5 to 30 and more preferably in a range of 10 to 30.

In Formula (1), from the viewpoint of the availability, it is preferable that R^{A} represents —CH₂CH₂—.

Specific examples of the compound represented by Formula (1) include methyl gluceth-10, methyl gluceth-20, PPG-10 methyl glucose, and PPG-20 methyl glucose.

Further, a commercially available product may be used as the compound represented by Formula (1), and specific examples thereof include MACBIOBRIDE (registered trademark) MG-10E, MACBIOBRIDE (registered trademark) MG-20E, MACBIOBRIDE (registered trademark) MG-10P, and MACBIOBRIDE (registered trademark) MG-20P (all manufactured by NOF Corporation), Glucam E-10, Glucam E-20, Glucam P-10, and Glucam P-20 (all manufactured by The Lubrizol Corporation), and NIKKOL (registered trademark) BMG-10 and NIKKOL (registered trademark) BMG-20 (both manufactured by Nikko Chemicals Co., Ltd.).

Next, the compound represented by Formula (2) will be described.

H—(OR^{B})ₘ—(OR^{C})ₙ—OH (2)

In Formula (2), R^{B} represents —CH₂CH₂—, and R^{C} represents or —CH₂CH₂CH₂—. m representing an average addition molar number of (OR^{B}) and n representing an average addition molar number of (OR^{C}) are each independently in a range of 0 to 200, and m+n is in a range of 3 to 200.

From the viewpoints of the availability and the handleability of the compound represented by Formula (2), m+n in Formula (2) is preferably in a range of 3 to 100 and more preferably in a range of 6 to 75.

From the viewpoint of the general purpose of the cosmetic, it is preferable that n in Formula (2) represents 0, in other words, it is preferable that the compound is a compound represented by H—(O—CH₂CH₂)ₘ—OH.

Specific examples of the compound represented by Formula (2) include PEG-4, PEG-6, PEG-8, PEG-12, PEG-20, PEG-8, PEG-32, PEG-40, PEG-75, PEG-150, PEG-200, PPG-12, PPG-17, PPG-20, PPG-34, and Poloxamer 105.

Further, a commercially available product may be used as the compound represented by Formula (2), and specific examples thereof include PEG #200, PEG #300, PEG #400, PEG #600, PEG #1000, PEG #1500, PEG #1540, PEG #2000, PEG #4000, PEG #6000, UNIOR (registered trademark) D-700, UNIOR (registered trademark) D-1000, UNIOR (registered trademark) D-1200, and UNIOR (registered trademark) D-2000 (all manufactured by NOF Corporation).

From the viewpoints of obtaining the water overflowing feeling at the time of application of the cosmetic onto the skin and suppressing the coating unevenness, the content of the (F) co-emulsifier (the ratio of the mass of the (F) co-emulsifier to the total mass of the water-in-oil type cosmetic) is preferably in a range of 0.1% by mass to 6% by mass, more preferably in a range of 0.5% by mass to 5% by mass, and still more preferably in a range of 0.5% by mass to 4% by mass with respect to the total amount of the water-in-oil type cosmetic.

The water-in-oil type cosmetic according to the present embodiment may contain only one or two or more kinds of the (F) co-emulsifiers.

[(G) Solid Particles Contained in Water Phase]

It is preferable that the water-in-oil type cosmetic according to the present embodiment contains (G) solid particles in the water phase.

In a case where the water phase contains the (G) solid particles, emulsified particles are easily broken at the time of application of the cosmetic onto the skin and the water overflowing feeling is easily obtained.

The expression of the “water phase contains the (G) solid particles” indicates that the (G) solid particles are present in the water phase which is a dispersed phase.

It is preferable that the (G) solid particles are dispersed in the water phase in order to easily exhibit the function of the solid particles.

The water phase may contain only one or two or more kinds of the (G) solid particles.

From the viewpoints of being easily contained in the water phase and allowing the emulsified state of the water-in-oil type cosmetic to be easily broken at the time of application of the cosmetic onto the skin, it is preferable that the (G) solid particle is a hydrophobic solid particle whose surface is subjected to a hydrophilic treatment or a hydrophobic solid particle to which the dispersibility in a water phase has been imparted. A method of using a dispersant or the like is exemplified as the method of imparting the dispersibility to hydrophobic solid particles in a water phase.

Specific examples of the (G) solid particles include titanium oxide whose surface is subjected to a hydrophilic treatment and particles of an organic ultraviolet absorbing agent to which the dispersibility in a water phase has been imparted.

Specific examples of titanium oxide whose surface is subjected to a hydrophilic treatment include commercially available products such as GT-10W and GT-10W2 (both manufactured by Sakai Chemical Industry Co., Ltd.), and WT-01 and WT-PF01 (both manufactured by Tayca Corporation).

Further, examples of the particles of the organic ultraviolet absorbing agent to which the dispersibility in a water phase has been imparted include commercially available products such as TINOSORB (registered trademark) M (manufactured by BASF SE) which is an aqueous dispersion of particles of an organic ultraviolet absorbing agent.

From the viewpoints of allowing the emulsified state of the water-in-oil type cosmetic to be easily broken at the time of application of the cosmetic onto the skin and the emulsion stability, the content of the (G) solid particles contained in the water phase (in other words, the ratio of the mass of the (G) solid particles contained in the water to the total mass of the water-in-oil type cosmetic) is preferably in a range of 0.01% by mass to 10% by mass, more preferably in a range of 0.03% by mass to 5% by mass, and still more preferably in a range of 0.05% by mass to 1.2% by mass with respect to the total amount of the water-in-oil type cosmetic.

In addition, a method of verifying the presence of the (G) solid particles in the water phase and the method of measuring the content of the (G) solid particles in the water phase are as follows.

In other words, the presence of the (G) solid particles in the water phase and the content thereof are acquired by centrifuging the measurement sample (that is, the water-in-oil type cosmetic), performing elemental analysis on the layer obtained by separation or extraction through the centrifugation, and performing analysis through comparison with the components (the component display of the cosmetic may be used) used for formulation of the measurement sample based on the results thereof. During the measurement, the measurement sample may be diluted with an organic solvent or water, and means required for the analysis such as a treatment or step of removing components other than the (G) solid particles may be appropriately used.

[(H) Thickening Polysaccharides]

It is preferable that the water-in-oil type cosmetic according to the present embodiment contains (H) thickening polysaccharides in the water phase.

In a case where the water phase contains the (H) thickening polysaccharides, the emulsion stability can be improved.

The water-in-oil type cosmetic according to the present embodiment may contain only one or two or more kinds of the (H) thickening polysaccharides.

Examples of the thickening polysaccharides include polysaccharides and derivatives thereof.

Examples the derivatives of polysaccharides include those obtained by bonding an alkyl group, a polyethylene oxide group, a polypropylene oxide group, or the like to some sugars in polysaccharides; and those obtained by bonding a single mannose, xylose, glucuronic acid, glucose, lactose, sucrose, or the like or a polymer having any of these sugars as a constitutional unit to some sugars in polysaccharides.

Preferred examples of the thickening polysaccharides include xanthan gum, hydroxyethyl cellulose, hydroxypropyl cellulose, hyaluronic acid, tremella fuciformis polysaccharide, salts of these, and derivatives of these. Among these, from the viewpoint of obtaining the water overflowing feeling at the time of application of the cosmetic onto the skin, at least one selected from the group consisting of hyaluronic acid and tremella fuciformis polysaccharide is preferable as the thickening polysaccharides.

Specific examples of the hyaluronic acid include commercially available products include FCH-200, FCH-150, FCH-120, FCH-80, FCH-60, and FCH-SU (all manufactured by Kikkoman Corporation), Hyaluronsan HA-Q, Hyaluronsan HA-M5070, Hyaluronsan HA-LQ, and Hyaluronsan HA-LQH (all manufactured by Kewpie Corporation).

Specific examples of the tremella fuciformis polysaccharide include commercially available products such as Tremoist-TP (manufactured by Nippon Fine Chemical Co., Ltd.).

From the viewpoints of improving the emulsion stability and exhibiting creaking which is a feeling of use specific to the polysaccharides, the content of the (H) thickening polysaccharides (in other words, the ratio of the mass of the (H) thickening polysaccharides to the total mass of the water-in-oil type cosmetic) is preferably in a range of 0.005% by mass to 0.3% by mass and more preferably in a range of 0.01% by mass to 0.2% by mass with respect to the total amount of the water-in-oil type cosmetic.

[Other Components]

The water-in-oil type cosmetic according to the present embodiment may contain components other than the above-described components (A) to (H) within the range where the effects according to the embodiment of the present invention are not impaired.

As other components, the components which can be blended into the cosmetic may be used, and examples thereof include moisturizers, touch improvers, ultraviolet absorbing agents other than the above-described titanium oxide and particles of the organic ultraviolet absorbing agent, water-soluble organic solvents, preservatives (such as phenoxyethanol and methyl paraben), pH adjusting agents, antioxidants, whitening agents, and flavoring agents.

Examples of the moisturizers and touch improvers include polyols of glycerin, 1,3-butylene glycol, propylene glycol, 3-methyl-1,3-butanediol, 1,3-propanediol, 2-methyl-1,3-propanediol, trimethylolpropane, pentaerythritol, hexylene glycol, diglycerin, polyglycerin, diethylene glycol, polyethylene glycol, dipropylene glycol, polypropylene glycol, and an ethylene glycol-propylene glycol copolymer and polymers thereof; glycol alkyl ethers such as diethylene glycol monoethyl ether (ethoxy diglycol), ethylene glycol monoethyl ether, ethylene glycol monobutyl ether, and diethylene glycol dibutyl ether; and (eicosanedioic acid-tetradecanedioic acid) polyglyceryl-10 esters.

Examples of the organic ultraviolet absorbing agents include benzoic acid-based ultraviolet absorbing agents such as paraaminobenzoic acid, paraaminobenzoic acid monoglycerin ester, N,N-dipropoxyparaaminobenzoic acid ethyl ester, N,N-diethoxyparaaminobenzoic acid ethyl ester, N,N-dimethylparaaminobenzoic acid ethyl ester, N,N-dimethylparaaminobenzoic acid butyl ester, and N,N-dimethylparaaminobenzoic acid ethyl ester; anthranilic acid-based ultraviolet absorbing agents such as homomenthyl-N-acetyl anthranilate; salicylic acid-based ultraviolet absorbing agents such as salicylic acid and a sodium salt thereof, amyl salicylate, menthyl salicylate, homomenthyl salicylate, octyl salicylate, phenyl salicylate, benzyl salicylate, and p-isopropanol phenyl salicylate; cinnamic acid-based ultraviolet absorbing agents such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, 2-ethylhexyl p-methoxy cinnamate (octyl paramethoxy cinnamate), 2-ethoxyethyl-p-methoxy cinnamate (cinoxate), cyclohexyl-p-methoxy cinnamate, ethyl-α-cyano-β-phenyl cinnamate, 2-ethylhexyl α-cyano-β-phenyl cinnamate (octocrylene), glyceryl mono-2-ethylhexanoyl-diparamethoxy cinnamate, and ferulic acid and derivatives thereof; benzophenone-based ultraviolet absorbing agents such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxyb enzophenone, 2-hydroxy-4-methoxybenzophenone (oxybenzone-3), 2-hydroxy-4-methoxy-4′-methylbenzophenone, 2-hydroxy-4-methoxybenzophenone-5-sulfonate, 4-phenylbenzophenone, 2-ethylhexyl-4′-phenyl-b enzophenone-2-carboxylate, 2-hydroxy-4-n-octoxybenzophenone, and 4-hydroxy-3-carboxybenzophenone; 3-(4′-methylbenzylidene)-d,l-camphor and 3-benzylidene-d,l-camphor; 2-phenyl-5-methylbeozoxazole; 2,2′-hydroxy-5-methylphenylbenzotriazole; 2-(2′-hydroxy-5′-t-octylphenyl) benzotriazole; 2-(2′-hydroxy-5′-methylphenyl)benzotriazole; dibenzalazine; dianisoylmethane; 5-(3,3-dimethyl-2-norbornylidene)-3-pentane-2-one; dibenzoylmethane derivatives such as t-butylmethoxydibenzoylmethane; octyl triazone; urocanic acid derivatives such as urocanic acid and ethyl urocanate; hydantoin derivatives such as 2-(2′-hydroxy-5′-methylphenyl) benzotriazole, 1-(3,4-dimethoxyphenyl)-4,4-dimethyl-1,3-pentanedione, 2-ethylhexyl dimethoxybenzylidenedioxoimidazolidine propionate; phenylbenzimidazole sulfonic acid, and terephthalylidene dicamphor sulfonic acid.

[Viscosity of Water-in-Oil Type Cosmetic]

From the viewpoints of the applicability to a tubular container, difficulty of dripping, and excellent spread, the viscosity of the water-in-oil type cosmetic according to the present embodiment at 25° C. is preferably 3000 mPa·s or greater and more preferably 4000 mPa·s or greater. Further, from the viewpoint of the viscosity stability, the viscosity thereof is still more preferably 15000 mPa·s and particularly preferably 20000 mPa·s or greater. Further, from the viewpoint of excellent spread, the viscosity of the water-in-oil type cosmetic according to the present embodiment at 25° C. is preferably 60000 mPa·s or less.

In the present disclosure, the viscosity measuring method of the water-in-oil type cosmetic at 25° C. is not limited as long as the viscosity is measured using a known viscosity measuring method.

For example, in the measurement of the viscosity, a value measured by performing stirring for 60 seconds at a rotor rotation speed of 6 rpm using a BL type viscometer (M4 rotor) can be used. As the BL type viscometer, for example, VISCOMETER TVB-10 (manufactured by Toki Sangyo Co., Ltd.) can be suitably used. Here, the BL type viscometer is not limited thereto.

[Proportion of Water Phase in Water-in-Oil Type Cosmetic]

From the viewpoints of easily obtaining the water overflowing feeling at the time of application of the cosmetic onto the skin and the emulsion stability, the proportion of the water phase which is a dispersed phase in the water-in-oil type cosmetic according to the present embodiment is preferably 45% by mass or greater, more preferably 50% by mass or greater, and still more preferably 55% by mass or greater with respect to the total amount of the water-in-oil type cosmetic.

From the viewpoint of exhibiting the emulsifying property, the emulsion stability, and the function of inorganic particles, the upper limit of the proportion of the water phase is preferably 85% by mass and more preferably 80% by mass with respect to the total amount of the water-in-oil type cosmetic.

The proportion of the water phase herein indicates the ratio of the total amount of components constituting the water phase (that is, the content of the water phase composition) to the total amount of all components (the total amount of the water-in-oil type cosmetic).

<Applications of Water-in-Oil Type Cosmetic>

Examples of the applications of the water-in-oil type cosmetic according to the present embodiment include makeup cosmetics, and specific examples thereof include base makeup cosmetics such as sunscreen cosmetics, makeup bases, and blemish balm (BB) creams, and the present embodiment is not limited to these.

<Method of Producing Water-in-Oil Type Cosmetic>

The method of producing the water-in-oil type cosmetic according to the present embodiment is not particularly limited, and the water-in-oil type cosmetic can be produced according to a known method of producing a water-in-oil type cosmetic.

For example, the water-in-oil type cosmetic according to the present embodiment can be produced by preparing the oil phase composition containing the components (A) to (D) and mixing the water phase composition containing the components (E) and (F) with the obtained oil phase composition.

From the viewpoints of the emulsion stability and suppression of the coating unevenness, it is more preferable that the water-in-oil type cosmetic according to the present embodiment is produced by respectively preparing the oil phase composition containing the components (A) to (D) and the water phase composition containing the components (E) to (G) and mixing the water phase composition with the obtained oil phase composition.

The emulsification method of mixing the oil phase composition with the water phase composition to obtain the water-in-oil type cosmetic according to the present embodiment as a water-in-oil type cosmetic is not particularly limited and can be performed according to a method of the related art.

Further, the conditions during the emulsification may be determined depending on the viscosity required for the water-in-oil type cosmetic according to the present embodiment and the size of the emulsified particle (water phase).

### EXAMPLES

Hereinafter, the present invention will be described in more detail based on examples, but is not limited to the following examples as long as the gist of the present invention is not impaired.

### Examples 1 to 14 and Comparative Examples 1 to 6

Each oil phase composition was obtained using components a to d listed in Tables 1 to 4.

Specifically, each oil phase composition was obtained by familiarizing the component d with a part of the component a in advance to prepare a uniformized paste, and dispersing the remaining component a, component b, and component c (at 1000 rpm (round per minute) for 10 minutes) in this paste so as to be mixed and uniformized.

Further, the water phase composition was obtained by heating the components e to i listed in Tables 1 to 4 as necessary and dispersing the components (1000 rpm for 10 minutes) so as to be mixed and uniformized.

Next, the emulsification was performed by adding the water phase composition to the oil phase composition little by little and appropriately changing the peripheral velocity and the emulsification time at 400 rpm to 1200 rpm using a homomixer.

The components and the contents thereof used in Examples 1 to 14 and Comparative Examples 1 to 6 are listed in the columns of the compositions of Tables 1 to 4. Further, “-” in the columns of the compositions indicates that the corresponding component is not contained.

※1: The content of the silicone oil in Tables 1 to 4 indicates the content of the silicone oil in the (A) oil agent, and the unit of “%” indicates “% by mass”.

※2: The proportion of the water phase in Tables 1 to 4 indicates the content ratio of the water phase composition to the total amount of all components, and the unit of “%” indicates “% by mass”.

In the present disclosure, the details of the components used in the examples and the comparative examples are as follows.

(Component a): oil agent containing 60% by mass or greater of silicone oil and having melting point of 20° C.
Dimethicone: KF-96A-5cs (manufactured by Shin-Etsu Chemical Co., Ltd.)
Cyclopentasiloxane: KF-995 (manufactured by Shin-Etsu Chemical Co., Ltd.)
(Caprylic acid/capric acid) triglyceride: COCONARD MT (manufactured by Kao Corporation)
Cetyl 2-ethylhexanoate: NIKKOL (registered trademark) CIO (manufactured by Nikko Chemicals Co., Ltd.)

(Component b) Silicone Gel
(Dimethicone/(PEG-10/15)) cross polymer: KSG-210 (product swollen by dimethicone and having 30% by mass of cross polymer, manufactured by Shin-Etsu Chemical Co., Ltd.)
(Dimethicone/vinyldimethicone) cross polymer: KSG-15 (product swollen by cyclopentasiloxane and having 5% by mass of cross polymer, manufactured by Shin-Etsu Chemical Co., Ltd.)

(Component c) Modified Silicone Surfactant Having HLB Value of Less than 6
PEG-9 polydimethylsiloxyethyl dimethicone: KF-6028 (HLB value of 4.5, manufactured by Shin-Etsu Chemical Co., Ltd.)
PEG-11 methyl ether dimethicone: KF-6011 (HLB value of 14.5, manufactured by Shin-Etsu Chemical Co., Ltd.)
Lauryl PEG-9 polydimethylsiloxyethyl dimethicone: KF-6038 (HLB value of 3.0, manufactured by Shin-Etsu Chemical Co., Ltd.)

(Component d) Inorganic Particles of at Least One Selected from Group Consisting of Titanium Oxide, Iron Oxide, and Mica
Surface-hydrophobicized titanium oxide: HXMT-100ZA (titanium oxide whose surface is treated with aluminum hydroxide and stearic acid and then further treated with 4-tert-butyl-4′-methoxydibenzoylmethane, manufactured by Tayca Corporation)

(Component f) Co-Emulsifier
PEG-6: PEG #300 (manufactured by NOF Corporation)
PEG-32: PEG #1540 (manufactured by NOF Corporation)
PEG-75: PEG #4000 (manufactured by NOF Corporation)
   Methyl gluceth-20: MACBIOBRIDE (registered trademark) MG-20E (manufactured by NOF Corporation)
   Methyl gluceth-10: MACBIOBRIDE (registered trademark) MG-10E (manufactured by NOF Corporation)

(Component g) Solid Particles Contained in Water Phase
TINOSORB M: TINOSORB (registered trademark) M (containing 50% by mass of methylene bisbenzotriazolyltetramethyl butyl phenol (MBBT), manufactured by BASF SE)
Fine particle titanium oxide dispersion: GT-10W2 (containing 50% by mass of fine particle titanium oxide, manufactured by Sakai Chemical Industry Co., Ltd.)
Pigment grade titanium oxide dispersion: WT-PF01 (containing 32% by mass of pigment grade titanium oxide, manufactured by Tayca Corporation).

(Component h) Thickening Polysaccharides
Xanthan gum: SAN-ACE C (manufactured by San-Ei Gen F. F. I., Inc.)
Tremella fuciformis polysaccharide: Tremoist-TP (manufactured by Nippon Fine Chemical Co., Ltd.)
Hyaluronic acid: Hyaluronsan HA-LF5-A (manufactured by Kewpie Corporation).

[Evaluation]

The water-in-oil type cosmetics of Examples 1 to 14 and Comparative Examples 2 to 6 were measured and evaluated in the following manners. The results are listed in Tables 1 to 4.

Further, since a water-in-oil type cosmetic was not able to be obtained in Comparative Example 1, only the evaluation of the emulsifying property among the following evaluations was performed. Therefore, “-” was noted in the columns of the measurement results and evaluation results other than the “emulsifying property”.

(Emulsifying Property)

Each water-in-oil type cosmetic of Examples 1 to 14 and Comparative Examples 1 to 6 was prepared according to the above-described procedures and allowed to stand, and it was confirmed whether separation or liquid separation occurred within 3 days, and the evaluation was performed based on the following evaluation standards. In the present disclosure, A was acceptable.

—Evaluation Standards—

A: The water-in-oil type cosmetic was able to be prepared, and separation or liquid separation did not occur in the prepared water-in-oil type cosmetic within 3 days.

B: The water-in-oil type cosmetic was not able to be prepared.

(Viscosity)

The viscosity of each water-in-oil type cosmetic of Examples 1 to 14 and Comparative Examples 2 to 6 at 25° C. was measured by stirring the cosmetic for 60 seconds at a rotor rotation speed of 6 rpm using a BL type viscometer (VISCOMETER TVB-10, manufactured by Toki Sangyo Co., Ltd., M4 rotor).

Further, the water-in-oil type cosmetic prepared within 5 hours was used for the measurement.

(Emulsion Stability)

Each water-in-oil type cosmetic of Examples 1 to 14 and Comparative Examples 2 to 6 was put into a sealed container and stored in a dark place at 50° C. for 28 days.

The appearance of the stored water-in-oil type cosmetic was visually confirmed.

The evaluation standards are as follows. In the present disclosure, 3 or higher is in an acceptable level.

—Evaluation Standards—

5: Separation did not occur and the emulsified state was maintained.

4: The viscosity was degraded, but separation did not occur, and the emulsified state was maintained.

3: Almost the entire water-in-oil type cosmetic maintained the emulsified state even though a trace amount of liquid separation occurred on the surface.

2: Most of the entire water-in-oil type cosmetic maintained the emulsified state even though a small amount of liquid separation occurred on the surface.

1: Liquid separation occurred, the liquid formed a layer, and the emulsified state was not maintained.

(Water Overflowing Feeling)

150 μl of each water-in-oil type cosmetic of Examples 1 to 14 and Comparative Examples 2 to 6 was applied and spread on the back of a hand of each of eight expert panelists, and the panelists evaluated whether the feeling of appearance of water droplets was able to be recognized.

The evaluation standards are as follows.

—Evaluation Standards—

5: Eight (all) out of eight members were able to recognize the feeling of appearance of water droplets.

4: Seven out of eight members were able to recognize the feeling of appearance of water droplets.

3: Six or five out of eight members were able to recognize the feeling of appearance of water droplets.

2: Four or three out of eight members were able to recognize the feeling of appearance of water droplets.

1: Two or less out of eight members were able to recognize the feeling of appearance of water droplets.

(Coating Unevenness)

In order to evaluate the level at which coating unevenness of the inorganic particles occurred after application of the cosmetic in actual use, after an oil-in-water type emulsion formulation (1) was applied to the inside of the forearm of each of eight expert panelists, 150 μl of each water-in-oil type cosmetic of Examples 1 to 14 and Comparative Examples 2 to 6 was applied and spread thereon, and the panelists determined whether the state thereof was one of the following three levels.

+++: The coating unevenness did not occur, and the cosmetic was uniformly applied by applying and spreading the cosmetic once.

++: The coating unevenness occurred, but the cosmetic was uniformly applied by applying and spreading the cosmetic two or more times.

+: The coating unevenness occurred, and the cosmetic was not uniformly applied until being dried.

The coating unevenness was evaluated based on the above-described determination results. The evaluation standards are as follows.

—Evaluation standards—

3: Six or more out of eight members determined the level as +++ or ++.

2: Five or four out of eight members determined the level as +++ or ++.

1: Three or less out of eight members determined the level as +++ or ++.

Here, The oil-in-water type emulsion formulation (1) used for evaluation of coating unevenness was prepared according to the following procedures.

[Preparation of Oil-in-Water Type Emulsion Formulation (1)]

The following components were mixed, and the mixture was stirred in a water bath at 80° C. for 10 minutes using a stirrer, thereby obtaining an oil phase composition.

—Composition of Oil Phase Composition—
Haematococcus pluvialis extract (trade name: ASTOTS-S (content of astaxanthin: 20% by mass), manufactured by Fujifilm Corporation): 0.01 parts by mass
Tomato extract (trade name: Lyc-O-Mato (registered trademark) 6% (content of lycopene: 6% by mass, manufactured by Sunbright Co., Ltd.): 0.008 parts by mass
Glyceryl stearate: 2 parts by mass
Polyglyceryl-6 stearate: 1 part by mass
PEG-60 glyceryl isostearate: 2 parts by mass
Lecithin (trade name: RESHION P, derived from soybeans, manufactured by Riken Vitamin Co., Ltd.): 0.5 parts by mass
Dimethicone (trade name: KF-96A-5cs, manufactured by Shin-Etsu Chemical Co., Ltd.): 2 parts by mass
Vaseline: 3 parts by mass
Behynyl alcohol: 3 parts by mass
Mixed tocopherol (trade name: RIKEN E OIL 800, manufactured by Riken Vitamin Co., Ltd.): 0.52 parts by mass
Cetyl ethyl hexanoate: 10 parts by mass
Pentaerythrityl tetraethyl hexanoate: 10 parts by mass

The following components were mixed, and the mixture was stirred in a water bath at 80° C. for 10 minutes using a stirrer, thereby obtaining an oil phase composition.

—Composition of Oil Phase Composition—
Evening primrose seed extract (trade name: Evening primrose seed PC, manufactured by Oryza Oil & Fat Chemical Co., Ltd.): 0.0025 parts by mass
Glycerin: 5 parts by mass
1,3-Butylene glycol: 5 parts by mass
Xanthan gum: 0.15 parts by mass
Methyl paraben: appropriate amount
Distilled water (name displayed on cosmetic: water): residual amount which becomes 100 parts by mass of oil-in-water type cosmetic in total

The water phase composition was stirred in a water bath at 80° C. using a homomixer, and the oil phase composition was gradually added to the water phase composition while being stirred. After completion of addition, the mixture was stirred for 10 minutes at a rotation speed of 3000 rpm using a homomixer (model type: TK ROBOMIX, manufactured by Primix Corporation) to obtain a coarse emulsion. The obtained coarse emulsion was cooled to 30° C., thereby obtaining an oil-in-water type emulsion formulation (1).

(Covering Powder)

A bioskin sheet (manufactured by Beaulax Co., Ltd.) with a stained portion simulating stain was coated with 0.6 mg/cm2 of each water-in-oil type cosmetic of Examples 1 to 14 and Comparative Examples 2 to 6, the sheet was allowed to stand for 5 minutes or longer, the stained portion and the unstained portion were measured using a color difference meter CR-700d (manufactured by Konita Minolta, Inc.) to obtain the brightness L*, a*, and b*, and the saturation C* obtained from each light source C.

The color difference ΔE between the value obtained from the stained portion and the value obtained from the unstained portion was calculated using the following equation.

Color difference (ΔE)={(ΔL*)²+(Δa*)²+(Δb*)²}^{1/2}

The evaluation standards are as follows.

—Evaluation Standards—

3: The color difference (ΔE) was 2 or less.

2: The color difference (ΔE) was greater than 2 and 4 or less.

1: The color difference (ΔE) was greater than 4.

(Applicability to Tubular Container)

A tubular polyethylene (PE) container having a volume of 30 ml and an opening with a diameter of 2.2 mm was filled with 30 g of each water-in-oil type cosmetic of Examples 1 to 14 and Comparative Examples 2 to 6. Thereafter, it was confirmed that the emulsion was dropped from a jet opening of the container in a case where the jet opening was turned down.

The evaluation standards are as follows.

A: Dropping of the emulsion from the jet opening was not confirmed.

B: Dropping of the emulsion from the jet opening was confirmed.

(Dropping from Finger)

75 μl of each water-in-oil type cosmetic of Examples 1 to 3 and Comparative Examples 2 to 6 was placed on fingers of two expert panelists, and two panelists evaluated whether the cosmetic was dropped from the fingers.

The evaluation standards are as follows.

—Evaluation Standards—

A: The emulsion was not dropped from the fingers of both panelists, and the shape of the emulsion was maintained from the state of the emulsion being placed on the fingers.

B: The emulsion was dropped from the fingers of one or two panelists.

(Excellent Spread)

150 μl of each water-in-oil type cosmetic of Examples 4 to 14 was applied and spread on the back of a hand of one expert panelist, and the panelist evaluated whether the water-in-oil type cosmetic followed the movement of a finger and the cosmetic easily spread on the skin.

The evaluation standards are as follows.

—Evaluation Standards—

3: The cosmetic had freshness and easily spread on the skin.

2: The panelist slightly had a creaking feeling, but the cosmetic easily spread on the skin, which was not problematic.

1: The panelist had a strong creaking feeling, the cosmetic easily spread on the skin, and the feeling of use was not good.

TABLE 1

| | | | | | | Comparative | Comparative |
|---|---|---|---|---|---|---|---|
| | | | Example 1 | Example 2 | Example 3 | Example 1 | Example 2 |
| Composition | a | Dimethicone | 12 | 10 | 8 | 12 | 12 |
| (parts by mass) | | (Caprylic acid/capric acid) triglyceride | — | 2 | — | — | — |
| | | Cetyl 2-ethyl hexanoate | — | — | 4 | — | — |
| | b | (Dimethicone/(PEG-10/15)) cross polymer | 3 | 3 | 3 | 3 | — |
| | | (Dimethicone/vinyldimethicone) cross polymer | 2 | 2 | 2 | 2 | — |
| | c | PEG-9 polydimethylsiloxyethyl dimethicone (HLB of 4.5) | 0.6 | 0.6 | 0.6 | — | 0.6 |
| | | PEG-11 methyl ether dimethicone (HLB of 14.5) | — | — | — | 0.6 | — |
| | d | Titanium oxide | 9 | 9 | 9 | 9 | 9 |
| | | Iron oxide | 1 | 1 | 1 | 1 | 1 |
| | e | Water | 66.1 | 66.1 | 66.1 | 66.1 | 71.1 |
| | f | PEG-6 | 1 | — | — | — | — |
| | | PEG-32 | — | 1 | — | 1 | 1 |
| | | Methyl gluceth-20 | — | — | 1 | — | — |
| | i | NaCl | 1 | 1 | 1 | 1 | 1 |
| | | Glycerin | 4 | 4 | 4 | 4 | 4 |
| | | Phenoxy ethanol | 0.3 | 0.3 | 0.3 | 0.3 | 0.3 |

| Viscosity at 25° C. [mPa · s] | | 35000 | 32000 | 16000 | — | 16000 |
|---|---|---|---|---|---|---|
| Content of silicone oil (※1) | | 100% | 88% | 75% | 100% | 100% |
| Proportion of water phase (※2) | | 72.4% | 72.4% | 72.4% | 72.4% | 77.4% |
| Evaluation | Emulsifying property | A | A | A | B | A |
| | Emulsion stability | 4 | 4 | 3 | — | 1 |
| | Water overflowing feeling | 3 | 3 | 3 | — | 4 |
| | Coating unevenness | 2 | 2 | 2 | — | 2 |
| | Covering powder | 2 | 2 | 2 | — | 2 |
| | Applicability to tubular container | A | A | A | — | A |
| | Dropping from finger | A | A | A | — | A |

| | | | Comparative | Comparative | Comparative | Comparative |
|---|---|---|---|---|---|---|
| | | | Example 3 | Example 4 | Example 5 | Example 6 |
| Composition | a | Dimethicone | 12 | 5 | 12 | 12 |
| (parts by mass) | | (Caprylic acid/capric acid) triglyceride | — | — | — | — |
| | | Cetyl 2-ethyl hexanoate | — | 7 | — | — |
| | b | (Dimethicone/(PEG-10/15)) cross polymer | 3 | 3 | 3 | 3 |
| | | (Dimethicone/vinyldimethicone) cross polymer | 2 | 2 | 2 | 2 |
| | c | PEG-9 polydimethylsiloxyethyl dimethicone (HLB of 4.5) | 3 | 0.6 | 0.1 | 0.6 |
| | | PEG-11 methyl ether dimethicone (HLB of 14.5) | — | — | — | — |
| | d | Titanium oxide | 9 | 9 | 9 | 9 |
| | | Iron oxide | 1 | 1 | 1 | 1 |
| | e | Water | 63.7 | 66.1 | 66.6 | 67.1 |
| | f | PEG-6 | — | — | — | — |
| | | PEG-32 | 1 | 1 | 1 | — |
| | | Methyl gluceth-20 | — | — | — | — |
| | i | NaCl | 1 | 1 | 1 | 1 |
| | | Glycerin | 4 | 4 | 4 | 4 |
| | | Phenoxy ethanol | 0.3 | 0.3 | 0.3 | 0.3 |

| Viscosity at 25° C. [mPa · s] | | 75000 | 12000 | 10000 | 25000 |
|---|---|---|---|---|---|
| Content of silicone oil (※1) | | 100% | 56% | 100% | 100% |
| Proportion of water phase (※2) | | 70.0% | 72.4% | 72.9% | 72.4% |
| Evaluation | Emulsifying property | A | A | A | A |
| | Emulsion stability | 5 | 2 | 1 | 4 |
| | Water overflowing feeling | 1 | 3 | 5 | 4 |
| | Coating unevenness | 2 | 2 | 2 | 1 |
| | Covering powder | 2 | 2 | 2 | 2 |
| | Applicability to tubular container | A | A | A | A |
| | Dropping from finger | A | A | A | A |

TABLE 2

| | | | Example 4 | Example 5 | Example 6 | Example 7 |
|---|---|---|---|---|---|---|
| Composition | a | Dimethicone | 5 | 5 | 5 | 5 |
| (parts by mass) | | Cyclopentasiloxane | 7 | 7 | 7 | 7 |
| | | Cetyl 2-ethyl hexanoate | — | 3 | — | — |
| | b | (Dimethicone/(PEG-10/15)) cross polymer | 2 | 2 | 2 | 2 |
| | | (Dimethicone/vinyldimethicone) cross polymer | 2 | 2 | 2 | 2 |
| | c | PEG-9 polydimethylsiloxyethyl dimethicone (HLB of 4.5) | 0.6 | 0.6 | 0.6 | 0.6 |
| | d | Titanium oxide | 9 | 9 | 9 | 9 |
| | | Iron oxide | 1 | 1 | 1 | 1 |
| | e | Water | 67.1 | 64.1 | 67.0 | 67.0 |
| | f | PEG-6 | — | — | 1 | 1 |
| | | PEG-75 | 1 | 1 | — | — |
| | i | NaCl | 1 | 1 | 1 | 1 |
| | | Glycerin | 4 | 4 | 4 | 4 |
| | | Phenoxy ethanol | 0.3 | 0.3 | 0.3 | 0.3 |
| | h | Xanthan gum | — | — | 0.1 | — |
| | | Tremella fuciformis polysaccharide | — | — | — | 0.1 |
| | | Hyaluronic acid | — | — | — | — |
| | | Hydroxyethyl cellulose | — | — | — | — |
| | | Hydroxypropyl cellulose | — | — | — | — |

| Viscosity at 25° C. [mPa · s] | | 26000 | 22000 | 24000 | 26000 |
|---|---|---|---|---|---|
| Content of silicone oil (※1) | | 100% | 84% | 100% | 100% |
| Proportion of water phase (※2) | | 73.4% | 70.4% | 73.4% | 73.4% |
| Evaluation | Emulsifying property | A | A | A | A |
| | Emulsion stability | 5 | 3 | 5 | 5 |
| | Water overflowing feeling | 3 | 3 | 3 | 4 |
| | Coating unevenness | 2 | 2 | 2 | 2 |
| | Covering powder | 2 | 2 | 2 | 2 |
| | Applicability to tubular container | A | A | A | A |
| | Excellent spread | 2 | 2 | 3 | 3 |

| | | | Example 8 | Example 9 | Example 10 |
|---|---|---|---|---|---|
| Composition | a | Dimethicone | 5 | 5 | 5 |
| (parts by mass) | | Cyclopentasiloxane | 7 | 7 | 7 |
| | | Cetyl 2-ethyl hexanoate | — | — | — |
| | b | (Dimethicone/(PEG-10/15)) cross polymer | 2 | 2 | 2 |
| | | (Dimethicone/vinyldimethicone) cross polymer | 2 | 2 | 2 |
| | c | PEG-9 polydimethylsiloxyethyl dimethicone (HLB of 4.5) | 0.6 | 0.6 | 0.6 |
| | d | Titanium oxide | 9 | 9 | 9 |
| | | Iron oxide | 1 | 1 | 1 |
| | e | Water | 67.0 | 67.0 | 67.0 |
| | f | PEG-6 | 1 | 1 | 1 |
| | | PEG-75 | — | — | — |
| | i | NaCl | 1 | 1 | 1 |
| | | Glycerin | 4 | 4 | 4 |
| | | Phenoxy ethanol | 0.3 | 0.3 | 0.3 |
| | h | Xanthan gum | — | — | — |
| | | Tremella fuciformis polysaccharide | — | — | — |
| | | Hyaluronic acid | 0.1 | — | — |
| | | Hydroxyethyl cellulose | — | 0.1 | — |
| | | Hydroxypropyl cellulose | — | — | 0.1 |

| Viscosity at 25° C. [mPa · s] | | 24000 | 21000 | 20000 |
|---|---|---|---|---|
| Content of silicone oil (※1) | | 100% | 100% | 100% |
| Proportion of water phase (※2) | | 73.4% | 73.4% | 73.4% |
| Evaluation | Emulsifying property | A | A | A |
| | Emulsion stability | 5 | 5 | 5 |
| | Water overflowing feeling | 4 | 3 | 3 |
| | Coating unevenness | 2 | 2 | 2 |
| | Covering powder | 2 | 2 | 2 |
| | Applicability to tubular container | A | A | A |
| | Excellent spread | 3 | 3 | 3 |

TABLE 3

| | | | Example 11 | Example 12 | Example 13 |
|---|---|---|---|---|---|
| Composition | a | Dimethicone | 5 | 5 | 5 |
| (parts by mass) | | Cyclopentasiloxane | 7 | 7 | 7 |
| | b | (Dimethicone/(PEG-10/15)) | 2 | 2 | 2 |
| | | cross polymer | | | |
| | | (Dimethicone/vinyldimethicone) | 2 | 2 | 2 |
| | | cross polymer | | | |
| | c | PEG-9 polydimethylsiloxyethyl | 0.6 | 0.6 | 0.6 |
| | | dimethicone (HLB of 4.5) | | | |
| | d | Titanium oxide | 9 | 9 | 9 |
| | | Iron oxide | 1 | 1 | 1 |
| | e | Water | 65.1 | 65.9 | 65.9 |
| | f | PEG-6 | 1 | 1 | 1 |
| | | PEG-32 | 1 | 1 | 1 |
| | i | NaCl | 1 | 1 | 1 |
| | | Glycerin | 4 | 4 | 4 |
| | | Phenoxy ethanol | 0.3 | 0.3 | 0.3 |
| | g | TINOSORB M | 1 | — | — |
| | | Fine particle titanium oxide | — | 0.2 | — |
| | | dispersion | | | |
| | | Pigment grade titanium oxide | — | — | 0.2 |
| | | dispersion | | | |
| Viscosity at 25° C. [mPa · s] | | | 24000 | 22000 | 23000 |
| Content of silicone oil (※1) | | | 100% | 100% | 100% |
| Proportion of water phase (※2) | | | 73.4% | 73.4% | 73.4% |
| Evaluation | | Emulsifying property | A | A | A |
| | | Emulsion stability | 4 | 4 | 4 |
| | | Water overflowing feeling | 4 | 5 | 5 |
| | | Coating unevenness | 3 | 3 | 3 |
| | | Covering powder | 2 | 2 | 2 |
| | | Applicability to tubular | A | A | A |
| | | container | | | |
| | | Excellent spread | 3 | 3 | 3 |

TABLE 4

| | | | Example 14 |
|---|---|---|---|
| Composition | a | Dimethicone | 4 |
| (parts by mass) | | Cyclopentasiloxane | 8 |
| | b | (Dimethicone/(PEG-10/15)) | 2 |
| | | cross polymer | |
| | | (Dimethicone/vinyldimethicone) | 2 |
| | | cross polymer | |
| | c | PEG-9 polydimethylsiloxyethyl | 0.6 |
| | | dimethicone (HLB of 4.5) | |
| | d | Surface-hydrophobicized | 4 |
| | | titanium oxide | |
| | | Titanium oxide | 5 |
| | | Iron oxide | 1 |
| | e | Water | 65.55 |
| | f | PEG-6 | 1 |
| | | PEG-32 | 1 |
| | i | NaCl | 1 |
| | | Glycerin | 4 |
| | | Phenoxy ethanol | 0.3 |
| | h | Tremellafuciformis | 0.05 |
| | | polysaccharide | |
| | g | Fine particle titanium | 0.5 |
| | | oxide dispersion | |
| Viscosity at 25° C. [mPa · s] | | | 29000 |
| Content of silicone oil (※1) | | | 100% |
| Proportion of water phase (※2) | | | 73.4% |
| Evaluation | | Emulsifying property | A |
| | | Emulsion stability | 5 |
| | | Water overflowing | 4 |
| | | feeling | |
| | | Coating unevenness | 3 |
| | | Covering powder | 3 |
| | | Applicability to | A |
| | | tubular container | |
| | | Excellent spread | 3 |

As listed in Tables 1 to 4, each water-in-oil type cosmetic of Examples 1 to 14 had an excellent emulsifying property and excellent emulsion stability while containing inorganic particles, the water overflowing feeling at the time of application of the cosmetic onto the skin was excellent, and the coating unevenness was reduced.

Further, it was found that each water-in-oil type cosmetic of Examples 1 to 14 had excellent covering power and excellent applicability to a tubular container, and the feeling of use based on the presence of dropping of the emulsion from the finger and excellent spread of the cosmetic was excellent.

In Comparative Example 1, since the cosmetic did not contain the (C) modified silicone surfactant having an HLB value of less than 6, an emulsion was not able to be obtained.

Further, in Comparative Example 2, a water-in-oil type cosmetic containing the (D) inorganic particles and having an excellent water overflowing feeling was obtained, but the emulsion stability thereof was degraded. Therefore, the cosmetic was not practically acceptable. The reason for this is considered that the water-in-oil type cosmetic of Comparative Example 2 did not contain the (B) silicone gel.

Further, in Comparative Example 3, a water-in-oil type cosmetic containing the (D) inorganic particles and having excellent emulsion stability was obtained, but the water overflowing feeling was not obtained. The reason for this is considered that the emulsified state of the water-in-oil type cosmetic of Comparative Example 3 was unlikely to be broken at the time of application of the cosmetic onto the skin because the content of the (C) modified silicone surfactant having an HLB value of less than 6 in the water-in-oil type cosmetic was extremely large.

Further, in Comparative Example 4, a water-in-oil type cosmetic containing the (D) inorganic particles and having an excellent water overflowing feeling was obtained, but the emulsion stability thereof was degraded. Therefore, the cosmetic was not practically acceptable. The reason for this is considered that the content of the silicone oil in the (A) oil agent in the water-in-oil type cosmetic of Comparative Example 4 was small.

Further, in Comparative Example 5, a water-in-oil type cosmetic containing the (D) inorganic particles and having an excellent water overflowing feeling was obtained, but the emulsion stability thereof was degraded. Therefore, the cosmetic was not practically acceptable. The reason for this is considered that the content of the (C) modified silicone surfactant having an HLB value of less than 6 in the water-in-oil type cosmetic of Comparative Example 5 was small.

Further, in Comparative Example 6, a water-in-oil type cosmetic containing the (D) inorganic particles and having excellent emulsion stability and an excellent water overflowing feeling was obtained, but the coating unevenness occurred. The reason for this is considered that the water-in-oil type cosmetic of Comparative Example 6 did not contain the (F) co-emulsifier.

Based on the comparison of Examples 6 to 10, it was found that the water overflowing feeling became excellent in a case of using tremella fuciformis polysaccharide or hyaluronic acid among the thickening polysaccharides.

Based on the comparison of Examples 4 with Examples 11 to 14, it was found that the water overflowing feeling became excellent in a case where the water phase contained solid particles (component g).

Based on the comparison of Examples 11 to 13, it was found that the water overflowing feeling became excellent depending on the kind of solid particles contained in the water phase.

### Example 15

[Actual Measurement and Evaluation of Water Overflowing Feeling]

First, a water-in-oil type cosmetic of Example 15 was obtained in the same manner as in Example 14 except that only the g component was replaced with water in the composition of Example 14.

Further, the water overflowing feeling of each water-in-oil type cosmetic of Example 14 and Example 15 at the time of application of the cosmetic onto the skin was quantitatively evaluated according to the following method using the average total area of liquid droplets as an index.

It is considered that the amount of water to spread on the skin is larger due to the shear force and the water overflowing feeling is stronger in the case of the cosmetic having a large average total area of liquid droplets.

(1) A bioskin sheet (manufactured by Beaulax Co., Ltd.) cut into a size of 5 cm×8 cm was set on Tribomaster TL201 Ts (manufactured by Trinity-Lab, Inc.).

(2) 15 μl of the obtained water-in-oil type cosmetic (Example 14 and Example 15) was placed on the bioskin sheet of (1).

(3) The bioskin sheet of (2) was allowed to reciprocate using a vibration type table (load of 20 g, speed of 100 mm/sec, distance of 50 mm).

(4) The process of (3) was repeated three times (third reciprocation), and the size (diameter) of liquid droplets which spread on the bioskin sheet was measured using a ruler.

(5) The processes of (1) to (4) were repeated three times, the average total area (unit: mm², average value of n=3 at the time of third reciprocation) of liquid droplets was acquired.

The measurement results of the average total area of the liquid droplets obtained using this method are listed in Table 5.

TABLE 5

| | Example 14 | Example 15 |
|---|---|---|
| Average total area of | 0.88 | 0.30 |
| liquid droplets | | |
| [mm², n = 3] | | |

As shown in Table 5, liquid droplets generated by breakage of emulsified particles due to the shear force were confirmed in both of the water-in-oil type cosmetics of Example 14 and Example 15. Further, it was found that the water overflowing feeling at the time of application of the cosmetic onto the skin was obtained from generation of liquid droplets.

Further, in the water-in-oil type cosmetic of Example 14 in which the component g was contained in the water phase, the average total area of liquid droplets was larger than that of the water-in-oil type cosmetic of Example 15 in which the component g was not contained in the water phase. Therefore, the water-in-oil type cosmetic of Example 14 had an excellent water overflowing feeling at the time of application of the cosmetic onto the skin.

### Prescription Example

<Preparation of Astaxanthin-Containing Emulsion Composition>

The following components were dissolved for 1 hour while being heated at 70° C., thereby obtaining a water phase composition A.
Sucrose stearic acid ester (manufactured by Mitsubishi-Chemicals Foods Corporation, Ryoto sugar ester S-1670): 3.3 g
Decaglyceryl monooleate (manufactured by Nikko Chemicals Co., Ltd.), NIKKOL (registered trademark) Decaglynl-OV): 6.7 g
Glycerin (alcohol): 45.0 g
Pure water: 30.0 g

The following components were dissolved for 1 hour while being heated at 70° C., thereby obtaining an oil phase composition A.
Haematococcus pluvialis extract (ASTOTS-S, manufactured by Fujifilm Corporation, content of astaxanthin: 20% by mass): 3.76 g
Mixed tocopherol (RIKEN E OIL 800, manufactured by Riken Vitamin Co., Ltd.): 0.96 g
Coconut oil (COCONARD MT, manufactured by Kao Corporation): 5.69 g
Lecithin (RESHION P, manufactured by Riken Vitamin Co., Ltd.): 1.0 g
Retinol palmitate-containing oil (RIKEN A Palmitate 1000 (E), manufactured by Riken Vitamin Co., Ltd., content of retinol palmitate: 55%): 3.6 g

The water phase composition A obtained in the above-described manner was stirred (10000 rpm) using a homogenizer (model name: HP93, manufactured by SMT Co., Ltd.) while maintaining the temperature at 70° C., and the oil phase composition A was added to the water phase composition A to obtain a pre-emulsion.

Next, the obtained pre-emulsion was cooled to approximately 40° C., and high-pressure emulsification was performed at a pressure of 245 MPa using Star Burst Mini HJP-25001 (manufactured by Sugino Machine Limited Co., Ltd.). After the high pressure emulsification, the emulsion was filtered using a microfilter having an average pore diameter of 1 μm to obtain astaxanthin-containing emulsion composition.

The obtained astaxanthin-containing emulsion composition was diluted with 1% by mass of milli Q water, the particle diameter of the dispersed particles was measured using a particle diameter analyzer FRAR-1000 (manufactured by Otsuka Electronics Co., Ltd.), and the value was 48.9 nm.

Further, the milli Q water indicates ultrapure water obtained by a milli Q water production device which is an ultrapure water production device (manufactured by Merch KGaA).

<Preparation of Ceramide Dispersion Composition>

The following components were stirred at room temperature for 1 hour, thereby preparing an oil phase composition B.
Ceramide 3 [ceramide compound]: 0.1 g
Ceramide 6 [Ceramide compound]: 0.1 g
Phytosphingosine: 0.07 g
Ethanol [water-soluble organic solvent]: 150 g
1 N hydrochloric acid (adjusted such that the pH measured immediately after dispersion was set to 7 or less)

The obtained oil phase composition B and water were micro-mixed using a collision type KM micromixer 100/100 at a ratio (mass ratio) of 1:7, thereby obtaining a ceramide dispersion composition.

Further, the conditions for using the micromixer are as follow.

—Microchannel—

Microchannel on Oil Phase Side

Cross-sectional shape/width/depth/length=rectangle/70 μm/100 μm/10 mm

Microchannel on Water Phase Side

Cross-sectional shape/width/depth/length=rectangle/490 μm/100 μm/10 mm

—Flow Rate—

Water was introduced to an outer ring at a flow rate of 21.0 ml/min and the oil phase composition B was introduced to an inner ring at a flow rate of 3.0 ml/min for micro mixing, thereby obtaining a pre-emulsion.

The solvent was removed from the obtained pre-emulsion using EVAPOR (CEP-lab) (manufactured by Okawara MFG. Co., Ltd.) until the concentration of ethanol reached 0.1% or greater, the resultant was concentrated and adjusted such that the concentration of the emulsion reached 2.0%, thereby obtaining a ceramide dispersion composition. The concentration of the emulsion here indicates the concentration based on the total amount of the solid content added to the oil phase.

### Example 16

Makeup Base

A makeup base (water-in-oil type cosmetic) having the following composition was prepared according to a method of the related art. As the result of evaluating the obtained makeup base according to the same method as in Example 1, the emulsifying property and the emulsion stability were excellent, the water overflowing feeling at the time of application of the cosmetic onto the skin was excellent, and the coating unevenness was reduced.

Further, each numerical value (% by mass) provided for each component indicates % by mass with respect to the total mass of the makeup base.
Dimethicone: 4% by mass
Cyclopentasiloxane: 8% by mass
KSG-210 (dimethicone swollen product of dimethicone/(PEG-10/15)) cross polymer, containing 30% by mass of cross polymer, manufactured by Shin-Etsu Chemical Co., Ltd.): 2% by mass
KSG-15 (cyclopentasiloxane swollen product of (dimethicone/vinyldimethicone) cross polymer, containing 5% by mass of cross polymer, manufactured by Shin-Etsu Chemical Co., Ltd.): 2% by mass
Lauryl PEG-9 polydimethylsiloxyethyl dimethicone (HLB value of 3.0): 0.3% by mass
PEG-9 polydimethylsiloxyethyl dimethicone (HLB value of 4.5): 0.5% by mass
Surface-hydrophobicized titanium oxide (HXMT-100ZA, manufactured by Tayca Corporation): 3% by mass
Titanium oxide: 4% by mass
Iron oxide: 1% by mass
Sodium chloride: 1% by mass
Ethanol: 1% by mass
Glycerin: 3% by mass
Phenoxy ethanol: 0.5% by mass
PEG-6: 1.5% by mass
PEG-32: 1.5% by mass
Tremella fuciformis polysaccharide: 0.02% by mass
Fine particle titanium oxide dispersion (GT-10W2, containing 50% by mass of fine particle titanium oxide, manufactured by Sakai Chemical Industry Co., Ltd.): 0.5% by mass
Astaxanthin-containing emulsion composition: 0.1% by mass
Composite powder pigment (HNB RED7: manufactured by Daito Kasei Kogyo Co., Ltd.): 0.01% by mass
Ceramide dispersion composition: 0.1% by mass
Water: residual amount

### Example 17

Sunscreen Agent

A sunscreen agent (water-in-oil type cosmetic) having the following composition was prepared according to a method of the related art. As the result of evaluating the obtained sunscreen agent according to the same method as in Example 1, the emulsifying property and the emulsion stability were excellent, the water overflowing feeling at the time of application of the cosmetic onto the skin was excellent, and the coating unevenness was reduced.

Further, each numerical value (% by mass) provided for each component indicates % by mass with respect to the total mass of the makeup base.
Dimethicone: 6% by mass
Cyclopentasiloxane: 4% by mass
Pentaerythrityl tetra-2-ethylhexanoate: 2% by mass
KSG-210 (dimethicone swollen product of dimethicone/(PEG-10/15)) cross polymer, containing 30% by mass of cross polymer, manufactured by Shin-Etsu Chemical Co., Ltd.): 3% by mass
KSG-15 (cyclopentasiloxane swollen product of (dimethicone/vinyldimethicone) cross polymer, containing 5% by mass of cross polymer, manufactured by Shin-Etsu Chemical Co., Ltd.): 0.5% by mass
Lauryl PEG-9 polydimethylsiloxyethyl dimethicone (HLB value of 3.0): 0.6% by mass
Titanium oxide: 4% by mass
Ethyl hexyl methoxy cinnamate: 2% by mass
t-Butylmethoxydibenzoylmethane: 2% by mass
Sodium chloride: 1% by mass
Ethanol: 1% by mass
Glycerin: 3% by mass
Phenoxy ethanol: 0.5% by mass
Methyl gluceth—10: 1% by mass
PEG-75: 1% by mass
Tremella fuciformis polysaccharide: 0.02% by mass
TINOSORB M: 2% by mass
Astaxanthin-containing emulsion composition: 0.5% by mass
Ceramide dispersion composition: 0.5% by mass
Water: residual amount

The disclosure of JP2017-103915A filed on May 25, 2017 is incorporated herein by reference.

In a case where all documents, patent applications, and technical standards described in the present specification are specified to be incorporated specifically and individually as cited documents, the documents, patent applications, and technical standards are incorporated herein in the same limited scope as the cited documents. The scope of the present invention is intended to be determined based on the following claims and the equivalents thereof.

What is claimed is:

1. A water-in-oil type cosmetic comprising:
an oil agent having a melting point of 20° C. or lower;
a silicone gel;
a modified silicone surfactant having a hydrophile-lipophile balance value of less than 6;
inorganic particles of at least one selected from the group consisting of titanium oxide, iron oxide, and mica;
water; and
a co-emulsifier,
wherein the oil agent having a melting point of 20° C. or lower comprises 60% by mass or greater of silicone oil,
a content of the modified silicone surfactant having a hydrophile-lipophile balance value of less than 6 is greater than 0.1% by mass and less than 1% by mass with respect to a total amount of the water-in-oil type cosmetic, and
the inorganic particles of at least one selected from the group consisting of titanium oxide, iron oxide, and mica are contained in an oil phase.

2. The water-in-oil type cosmetic according to claim 1, wherein the oil phase comprises the inorganic particles of at least one selected from the group consisting of titanium oxide, iron oxide, and mica at a content of 5% by mass to 30% by mass with respect to the total amount of the water-in-oil type cosmetic.

3. The water-in-oil type cosmetic according to claim 1, having a water phase comprising solid particles.

4. The water-in-oil type cosmetic according to claim 3, wherein the solid particles are hydrophobic solid particles a surface of each of which has been subjected to a hydrophilic treatment or hydrophobic solid particles to which dispersibility in the water phase has been imparted.

5. The water-in-oil type cosmetic according to claim 1, wherein the modified silicone surfactant having a hydrophile-lipophile balance value of less than 6 has a branched silicone chain in a structure thereof.

6. The water-in-oil type cosmetic according to claim 1, wherein the water phase comprises a thickening polysaccharide.

7. The water-in-oil type cosmetic according to claim 1, wherein the co-emulsifier is at least one compound selected from the group consisting of a compound represented by Formula (1) and a compound represented by Formula (2): 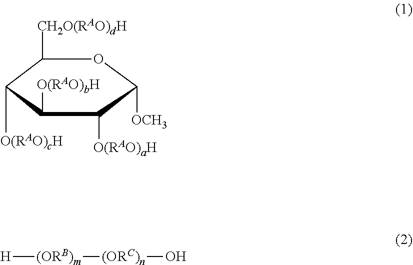
wherein in Formula (1), R^{A} represents —CH₂CH₂— or —CH₂CH₂CH₂—; a, b, c, and d respectively represent an average addition molar number of (R^{A}O) and are each independently in a range of 0 to 200; and a+b+c+d is in a range of 3 to 200; and
wherein in Formula (2), R^{B} represents —CH₂CH₂—; R^{C} represents —CH₂CH₂CH₂—; m represents an average addition molar number of (OR^{B}); n represents an average addition molar number of (OR^{C}); n and m are each independently in a range of 0 to 200; and m+n is in a range of 3 to 200.

8. The water-in-oil type cosmetic according to claim 1, wherein the co-emulsifier is at least one compound selected from the group consisting of a compound represented by Formula (2):
H—(OR^{B})ₘ—(OR^{C})ₙ—OH (2)
wherein in Formula (2), R^{B} represents —CH₂CH₂—; R^{C} represents or —CH₂CH₂CH₂—; m represents an average addition molar number of (OR^{B}); n represents an average addition molar number of (OR^{C}); n and m are each independently in a range of 0 to 200; and m+n is in a range of 3 to 200.

9. The water-in-oil type cosmetic according to claim 8, wherein, in the compound represented by Formula (2), m+n is in a range of 6 to 75.

10. The water-in-oil type cosmetic according to claim 1, wherein the silicone oil comprises at least one selected from the group consisting of dimethylpolysiloxane and decamethylcyclopentasiloxane.

11. The water-in-oil type cosmetic according to claim 1, wherein the oil agent having a melting point of 20° C. or lower comprises 75% by mass to 100% by mass of the silicone oil.

12. The water-in-oil type cosmetic according to claim 1, wherein the silicone gel comprises at least one selected from the group consisting of a polyether-modified silicone gel and a silicone three-dimensional crosslinked product.

13. The water-in-oil type cosmetic according to claim 1, wherein a content of the silicone gel is in a range of 0.25% by mass to 4.5% by mass with respect to the total amount of the water-in-oil type cosmetic.

14. The water-in-oil type cosmetic according to claim 1, having a viscosity at 25° C. of 3000 mPa·s or greater.
